SEQUENCE LISTING (4 sequences)

SEQ ID NO: 1 - protein, 226 aa, Pleurotus salmoneostramineus
MSLTLSPLPPLSNDIYPIGRNSLGNLMTATEKAKELPQEDKSAAQFQATSQESYKSAVSQTTKESPSASLAKFCKEAETAYPALYKAIQANDSASAKELAKSIASKLTEVATSAGNVAQAYNQGAAKAQEGQKLMKSALPGSHPVKDSVDDALQYLSPAAQVFTSMQSSLNESAKNVVAAADKVGKVPANQIASEDSGEAIANAWAKLGVKATAQAEAYNKWQGNQ

SEQ ID NO: 2 - protein, 254 aa, Deinococcus geothermalis
MDYRTVFRLDGACAAVTGAGSGIGLEICRAFAASGARLILIDREGAALDRAAEELGAAVASRIVADVTDAEAMTAAAAAAEAVAPVSILVNSAGIARLHDALETDDATWRQVMAVNVDGMFWASRAFGRAMVARGAGAIVNLGSMSGTIVNRPQFASSYMASKGAVHQLTRALAAEWAGRGVRVNALAPGYVATEMTLKMRERPELFGTWLDMTPMGRCGEPSEIAAAALFLASPAASYVTGAILAVDGGYTVW

SEQ ID NO: 3 - protein, 1068 aa, Streptomyces cattleya
MSPTPHSASGTTGAAAATPGAASPAPPVPVADISDTGFGTTPIQQAMALAREHGPVFRRRFGTFESLLVGSVDAVTELCDDERFVKAVGPVLTNVRQIAGDGLFTAYNDEPNWAKAHDILLPAFALSSMHTYHPTMLRVAKRLIAAWDTALADGAPVDVADDMTRMTLDTIGLAGFGYDFGSFRRGEPHPFVAAMVRGLLHSQALLSRKADDGVDHSAADEAFRADNAYLAQVVDEVIEARRASGETGTDDLLGLMLGAPHPSDGTPLDAANIRNQVITFLIAGHETTSGALSFALYYLAKNPAVLRRAQAEVDALWGDDPDPEPDYTDVGRLTYVRQVLNEALRLWPTAAAFGRQAVTDTVLDGRVPMRAGDTALVLTPVLHRDPVWGDNVEAFDPERFSPEREAARPVHAFKPFGTGERACIGRQFALHEAVMLLGMLIHRYRFLDHADYRLRVRETLTLKPDGFTLKLARRTSADRVRTVASRAAEGTAGQDAGLPTTARPGTTLTVLHGSNLGACREFAAGLADLGERCGFETTVAPLDAYRAGDLPRTSPVVVVAASYNGRPTDDAAGFVSWLEQAGPGAADGVRYAVLGVGDRNWAATYQKVPTLIDERLAECGATRLLERAAADAAGDLAGTVRGFGEALRRALLAEYGDPDSVGAVAGAEDGYEVTEVTGGPLDALAARHEVVAMTVTETGDLADLTHPLGRSKRFVRLALPDGATYRTGDHLAVLPANDPALVERAARLLGADPDTVLGVRARRPGRGTLPVDRPVTVRELLTYHLELSDPATAAQIAVLADRNPCPPEQAELKKLAPGRASVLDLVERYPALTGRLDWPTVLGTLLPQIRIRHYSVSSSPAVSPGHVDLMVSLLEADGRRGTGSGHLHRVRPGDVVYARVAPCREAFRIAAGDEVPVVMVAAGTGLAPFRGAVADRVALRSAGRELAPALLYFGCDHPEVDFLHAAELRGAEAAGAVSLRPAFSAAPDGDVRFVQHRIAAEADEVWSLLKGGARVYVCGDGSRMAPGVREAFTALYASRTGATAEQAAGWLADLVARGRYVEDVYAAG

SEQ ID NO: 4 - protein, 20 aa, Artificial Sequence
  synthetic purification tag
MGSSHHHHHHSSGLVPRGSH

(19) United States
(12) Patent Application Publication
(10) Pub. No.: US 2024/0349762 A1
(43) Pub. Date: Oct. 24, 2024

(54) PIGMENT FOR MEAT SUBSTITUTE COMPOSITIONS

(71) Applicant: Cargill, Incorporated, Wayzata, MN (US)

(72) Inventors: Jamie Koehler, Minneapolis, MN (US); Nandita Kohli, Plymouth, MN (US); Hans H. Liao, Plymouth, MN (US)

(73) Assignee: Cargill, Incorporated, Wayzata, MN (US)

(21) Appl. No.: 18/555,969

(22) PCT Filed: Apr. 14, 2022
(86) PCT No.: PCT/US2022/024749

§ 371 Date: Oct. 18, 2023

Related U.S. Application Data
(60) Provisional application No. 63/176,575, filed on Apr. 19, 2021.

(51) Int. Cl. A23L 5/43; A23J 1/00; A23J 3/22; A23L 31/00

(52) U.S. Cl. CPC A23L 5/43; A23J 1/006; A23J 3/227; A23L 31/00

(57) ABSTRACT

Disclosed herein are pigment compositions for meat substitutes and meat substitutes including such pigment compositions. The pigment compositions include a thermolabile pink oyster mushroom pink chromogenic protein. The pigment compositions provide a pink and/or red color to a meat substitute composition that transitions to colorless or a brown color after cooking.

### CROSS-REFERENCE TO RELATED APPLICATIONS

This application claims priority to U.S. Provisional Application No. 63/176,575, filed Apr. 19, 2021, which is incorporated herein by reference in its entirety.

### REFERENCE TO A SEQUENCE LISTING SUBMITTED VIA EFS-WEB

The content of the ASCII text file of the sequence listing named “PT1048_ST25.txt” which is 13.6 kb in size created on Apr. 11, 2022 and electronically submitted vis EFS-Web herewith the application is incorporated by reference in its entirety.

### BACKGROUND

Demand for plant-based meat substitutes is increasing for a variety of reasons. Many consumers prefer meat substitute options that perform most similarly to animal meat, including wanting the color of the meat substitute to be comparable to animal meat color before and after cooking. Accordingly, there is a need for a pigment that can provide color to a meat substitute that is the same or similar to that of natural animal meat. A pigment derived from natural sources that can transition in color when the meat substitute is cooked is particularly desirable.

### SUMMARY

The present disclosure provides compositions comprising a pink oyster mushroom extract in an amount effective for increasing the red or pink color of a meat substitute. The pink oyster mushroom extract may be an aqueous extract of Pleurotus djamor or Pleurotus salmoneostramineus. The composition may be a pigment composition.

The present disclosure also provides compositions comprising a pink chromogenic protein (PCP) in an amount effective for increasing the red color of a meat substitute. The PCP may have an absorbance maximum between 450 nm and 600 nm and may be from a Pleurotus species. The PCP may be from Pleurotus djamor or Pleurotus salmoneostramineus. The composition may additionally comprise indol-3-one in a molar ration between 0.5:1 to 2:1 with the PCP. The PCP may comprise a sequence at least 75%, at least 80%, at least 85%, at least 90%, at least 95%, at least 98%, or at least 99% identical to SEQ ID NO:1. The composition may be a pigment composition.

For the compositions (e.g., pigment compositions) described herein, the red color of the composition may be decreased when heated at 130° C. for 2 minutes. When heated at 130° C. for 2 minutes the a* value of L*a*b* colorimetry of the composition may decrease by at least 5, 10, 15, 20, 25, 30, 35, 40, 45, or 50%. When the composition is heated at 130° C. for 2 minutes, absorbance of light at a wavelength of 496 nm may decrease relative to the absorbance prior to heating.

For example, the disclosure provides pigment compositions comprising prink oyster mushroom, an extract (e.g., an aqueous extract) of pink oyster mushroom, and/or a pink chromogenic protein (PCP).

The disclosure also provides a meat substitute comprising a non-meat protein and (i) a pigment composition comprising a pink oyster mushroom extract; (ii) pink oyster mushroom; and/or (iii) a pigment composition comprising a PCP. The pigment composition may comprise an aqueous extract of Pleurotus djamor or Pleurotus salmoneostramineus. The meat substitute composition may comprise pink oyster mushroom that is chopped, ground, pureed, crushed, or dried pink oyster mushroom. The meat substitute may comprise PCP with an absorbance maximum between 450 nm and 600 nm and that is from a Pleurotus species. The PCP may be from Pleurotus djamor or Pleurotus salmoneostramineus. The meat substitute may additionally comprise indol-3-one in a molar ration between 0.5:1 to 2:1 with the PCP. The PCP may comprise a sequence at least 75%, at least 80%, at least 85%, at least 90%, at least 95%, at least 98%, or at least 99% identical to SEQ ID NO:1. The non-meat protein may be a plant-based protein selected from the group consisting of pea protein, soy protein, corn protein, and wheat protein. The non-meat protein may be a fungal-derived mycoprotein. The meat substitute may comprise 0.01% to 6%, 0.05% to 5%, 0.1% to 3%, or 0.5% to 2% by weight of indol-3-one bound PCP. The red color of the meat substitute may decrease after cooking. When heated at 130° C. for 2 minutes the a* value of L*a*b* colorimetry of the meat substitute may decrease by at least 5, 10, 15, 20, 25, 30, 35, 40, 45, or 50%.

The disclosure also provides a method for increasing the red color of a meat substitute, comprising adding (i) a pink oyster mushroom extract; (ii) pink oyster mushroom; and/or (iii) a PCP to a meat substitute comprising a non-meat protein. The pink oyster mushroom extract may be an aqueous extract of Pleurotus djamor or Pleurotus salmoneostramineus. The pink oyster mushroom may be chopped, ground, pureed, crushed, or dried pink oyster mushroom. The PCP may have an absorbance maximum between 450 nm and 600 nm and be from a Pleurotus species. The PCP may be from Pleurotus djamor or Pleurotus salmoneostramineus. The PCP may be added as part of a pigment composition comprising the PCP and indol-3-one. The pigment composition may comprise indol-3-one and PCP in a molar ratio between 0.5:1 and 2:1. The PCP may comprise a sequence at least 75%, at least 80%, at least 85%, at least 90%, at least 95%, at least 98%, or at least 99% identical to SEQ ID NO:1.

The disclosure also provides a method for decreasing red color in a cooked meat substitute, comprising cooking a meat substitute comprising a non-meat protein and (i) a pink oyster mushroom extract; (ii) pink oyster mushroom; and/or (iii) a PCP, whereby the red color of the cooked meat substitute is reduced relative to the red color of the meat substitute prior to cooking. The pink oyster mushroom extract may be an aqueous extract of Pleurotus djamor or Pleurotus salmoneostramineus. The pink oyster mushroom may be chopped, ground, pureed, crushed, or dried pink oyster mushroom. The PCP may have an absorbance maximum between 450 nm and 600 nm and be from a Pleurotus species. The PCP may be from Pleurotus djamor or Pleurotus salmoneostramineus. The PCP may be added as part of a pigment composition comprising the PCP and indol-3-one. The pigment composition may comprise indol-3-one and PCP in a molar ratio between 0.5:1 and 2:1. The PCP may comprise a sequence at least 75%, at least 80%, at least 85%, at least 90%, at least 95%, at least 98%, or at least 99% identical to SEQ ID NO: 1. When heated at 130° C. for 2 minutes the a* value of L*a*b* colorimetry of the meat substitute may decrease by at least 5, 10, 15, 20, 25, 30, 35, 40, 45, or 50%. The non-meat protein may be or may comprise a plant-based protein selected from the group consisting of pea protein, soy protein, corn protein, and wheat protein. The non-meat protein may be or may comprise a fungal-derived mycoprotein. The meat substitute may comprise 0.01% to 6%, 0.05% to 5%, 0.1% to 3%, or 0.5% to 2% by weight of indol-3-one bound PCP.

The disclosure also provides a recombinant host cell capable of producing indol-3-one and a pink chromogenic protein, the cell comprising: (i) an exogenous nucleic acid sequence encoding a polypeptide at least 80% identical to SEQ ID NO:1; (ii) a polynucleotide encoding a tryptophan feedback-insensitive DAHP synthase; (iii) an exogenous polynucleotide encoding the CYP102A cytochrome P450 monooxygenase from Streptomyces cattleya; and (iv) an exogenous polynucleotide encoding an indoxyl dehydrogenase or reductase, wherein one or more tryptophan biosynthetic genes are overexpressed in the recombinant host cell and the recombinant host cell produces a PCP-indol-3-one complex. The PCP-indol-3-one complex may have an absorbance maximum of about 496 nm. The recombinant host cell may comprise a deletion or disruption of native DAHP synthase gene.

The disclosure also provides a recombinant host cell capable of producing indol-3-one and a pink chromogenic protein, the cell comprising: (i) an exogenous nucleic acid sequence encoding a polypeptide at least 80% identical to SEQ ID NO:1; (ii) an exogenous polynucleotide encoding a tryptophanase; (iii) an exogenous polynucleotide encoding a CYP102A cytochrome P450 monooxygenase from Streptomyces cattleya; and (iv) an exogenous polynucleotide encoding an indoxyl dehydrogenase or reductase, wherein the recombinant host cell produces a PCP-indol-3-one complex. The PCP-indol-3-one complex may have an absorbance maximum of about 496 nm.

The recombinant host cells described herein may be an Escherichia coli, Bacillus subtilis, Fusarium venenatum, Pichia pastoris, Saccharomyces cerevisiae, Kluyveromyces lactis, Yarrowia lipolytica, Trichomderma reesei, Issatchenkia orientalis, Aspergillus niger, Agaricus bisporus, Lentinula edodes, Volvariella volvacea, Pisum sativum, Zea mays, Glycine max or Triticum sp. cell. The recombinant host cell may be an Escherichia coli, Bacillus subtilis, Fusarium venenatum, Pichia pastoris, Saccharomyces cerevisiae, Kluyveromyces lactis, Yarrowia lipolytica, Trichomderma reesei, Issatchenkia orientalis, or Aspergillus niger cell.

Also provided is a meat substitute comprising a recombinant cell described here in a non-meat protein. Also provided is a method for preparing a meat substitute with increased red color, the method comprising: combining a non-meat protein and a PCP-indol-3-one complex produced by a recombinant host cell described herein to form a meat substitute with increased red color compared to a meat substitute prepared without the PCP-indole-3-one complex. The non-meat protein may be combined with a recombinant host cell described herein and comprising the PCP-indol-3-one complex. The method may additionally comprise the step of isolating the PCP-indol-3-one complex from the recombinant host cell prior to combining with the non-meat protein. The meat substitute may comprise 0.01% to 6%, 0.05% to 5%, 0.1% to 3%, or 0.5% to 2% by weight of the PCP-indol-3-one complex.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed in the present document.

FIG. 1 shows photos of pink oyster mushroom.

FIG. 2 shows a photo of chopped pink oyster mushroom before (right) and after (left) heating on a 130° C. hot plate for 1 minutes.

FIG. 3 shows a pink oyster mushroom extract in water separated by centrifugation from a puree of pink oyster mushroom in water.

FIG. 4 shows from left to right a puree of pink oyster mushroom in water, the pink oyster mushroom extract of FIG. 3, the pink oyster mushroom extract after heating on a 130° C. hot plate for 40 seconds, and a puree of pink oyster mushroom in water after heating on a 130° C. hot plate for 1 minutes.

FIG. 5 shows a Hunter colorimetry reflectance plot for the pink oyster mushroom puree before heating (bottom line) and after heating at 130° C. for 1 minutes (top line).

FIGS. 6A and 6B show absorbance data for the pink oyster mushroom extract (supernatant separated from puree) before (top line) and after heating (bottom line) on a 130° C. hot plate for 40 seconds. FIG. 6A shows the full spectrum of absorbance data collected and FIG. 6B highlights the absorbance range from about 348.8 nm to 648.8 nm.

FIG. 7 shows a comparison between an initial aqueous pink oyster mushroom extract (left) and an 8× concentrated extract.

FIG. 8 shows a comparison of the absorbance spectrum of the initial aqueous pink oyster mushroom extract (bottom line) and the 8× concentrate (top line).

FIGS. 9A and 9B show a comparison of the color of beef (left) and the pink oyster mushroom extract in a meat-substitute composition (right). FIG. 9A shows a comparison in the raw color, and FIG. 9B shows a comparison following heating each sample on a 130° C. hotplate for 1 minutes.

FIG. 10 shows absorbance spectra of Fractions A-H as outlined in Example 4.

FIG. 11 shows absorbance spectra of Fractions A and E of Example 4 with and without trypsin digest.

FIG. 12 shows the visual appearance of Fraction A with (G5) and without (G6) trypsin digest and Fraction E with (G7) and without (G8) trypsin digest. Both samples of Fraction E were diluted 1:2 in 50 mM HEPES aqueous solution.

FIG. 13 shows the change in absorbance between the trypsin digested and undigested samples of Fractions A and E as outlined in Example 4.

FIG. 14 shows a photo of Fraction A with (H11) and without (H10) EDTA treatment and a sample of the EDTA treatment condition alone (H12) without any pink oyster mushroom fraction.

FIG. 15 show absorbance spectra of Fraction A with and without EDTA treatment and the EDTA condition alone.

FIG. 16 shows the change in absorbance between the EDTA treated and untreated Fraction A samples.

FIGS. 17A and 17B show unstrained (17A) and stained (17B) native gel electrophoresis results as outlined in Example 4.

FIG. 18 shows samples of (from left to right) crude oyster mushroom extract, concentrated crude oyster mushroom extract, acetone extracted soluble fraction from pink oyster containing indol-3-one, isolated recombinant PsPCP, and the recombinant PsPCP in combination with the acetone extraction containing indol-3-one.

FIG. 19 shows absorbance spectra of the samples shown in FIG. 18 and outlined in Example 6.

FIG. 20 shows the biosynthetic pathway of for the production of indol-3-one (also referred to as indolone) as described in Example 7.

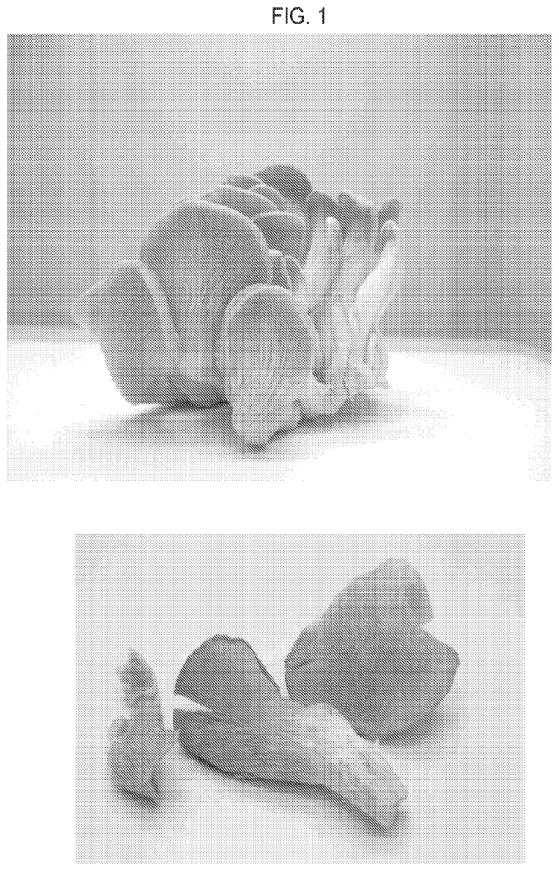

### DETAILED DESCRIPTION

Described herein are pigment compositions for meat substitutes that contain a thermolabile pink chromogenic protein. Thermolabile PsPCP, the pink chromogenic protein from Pleurotus salmoneostramineus, and other Pleurotus sp. PCPs may be used in a pigment composition having a similar pink/red color to raw animal meat before cooking and are susceptible to degradation during heating. This degradation of the pigment composition causes the pigment to have a substantially reduced color or become colorless after heating. Accordingly, meat substitutes containing an effective amount of this pigment composition will transition from a pink/red color when raw to a brown or less red color when cooked. In an aspect, the brown color occurs because the pigment composition in the meat substitute becomes at least partially colorless during heating, which allows the brown color resulting from Maillard reactions involving other components of the meat substitute to become more visible than with other pigments used for meat substitutes.

Unless defined otherwise, all technical and scientific terms used herein have the same meaning as commonly understood by one skilled in the art to which this invention belongs. As used herein, each of the following terms has the meaning associated with it as defined below.

As used herein, the terms “meat substitute” and “meat substitute composition” are used interchangeably and refer to compositions that mimic the general appearance, nutritional content, and/or taste of natural animal meat or natural animal meat compositions without containing as the majority component tissues or cells from a whole, living vertebrate animal. For example, the meat substitute may be free of, or contain as a minor component, naturally-occurring animal muscle, adipose, or satellite cells from muscle tissues harvested from a whole vertebrate animal (e.g., a cow, a sheep, a pig, a chicken, a turkey, etc.). In some aspects, the meat substitute is free of any animal cells, e.g., any in vivo derived or in vitro cultured animal cells.

The meat substitutes and meat substitute compositions described herein include non-meat proteins, plant-based proteins (e.g., pea protein, soy protein, wheat protein, chickpea protein, corn protein, and the like), fungal-based proteins (e.g., mycoproteins derived from fungi such as Fusarium venenatum and the like), in vitro cultured animal cells (e.g., cultured muscle cells, satellite cells, adipose cells, and the like), insect proteins, or combinations thereof. The meat substitute can comprise plant-based proteins including, but not limited to, pea protein, soy protein, wheat protein, chickpea protein, and corn protein. The meat substitute can comprise fungal based proteins including, but not limited to, mycoproteins from Fusarium venenatum. The meat substitute can comprise a fungal extract, including, but not limited to a Fusarium venenatum and/or a Pleurotus sp. extract (e.g., Pleurotus salmoneostramineus, Pleurotus djamor, and the like). The meat substitute can comprise in vitro cultured animal cells including, but not limited to, muscle cells, satellite cells, and adipose cells grown, differentiated, and propagated using, for example, fermentation, a bioreactor, scaffold-seeded cell culture, or other artificial methods. The meat substitute can comprise a combination of two or more of plant-based protein, fungal-based proteins, insect proteins and in vitro cultured animal cells. For example, a meat substitute may include a pea protein and a fungal mycoprotein, a soy protein and a cultured bovine muscle cell, a cultured avian adipocyte and a fungal mycoprotein, or any other combination of plant-base protein, fungal-based protein, insect proteins, and in vitro cultured animal cells.

In some aspects, the meat substitute comprises plant-based proteins, fungal-based proteins, or combinations thereof and is free of any animal-based proteins or cells. In some aspects, the meat substitute comprises plant-based proteins, fungal-based proteins, insect proteins, and combinations thereof and is free of and any vertebrate animal-based cells or proteins. In some aspects, the meat substitute comprises plant-based proteins and is free of non-pigmented fungal-based, insect, or animal-based cells or proteins. In some aspects, the meat substitutes comprise fungal-based proteins and is free of plant-based, insect, and animal-based cells and proteins. In some aspects, the meat substitute comprises insect proteins and is free of plant-based, non-pigmented fungal-based, and animal-based cells and proteins. In some aspects, the meat substitute comprises in vivo cultured animal cells and is free of plant-based proteins, non-pigmented fungal-based proteins, insect proteins, and in vivo whole animal derived tissues, cells, and proteins.

In some aspects, the meat substitute can mimic a beef product, e.g., ground beef, steak, beef jerky, beef ribs, beef patties, beef sausages, and the like. In some aspects, the meat substitute can mimic a pork product, e.g., ground pork, pork chops, ham, smoked pork, bacon, pork sausage, pork patties, pork ribs, and the like. In some aspects, the meat substitute can mimic a chicken product, e.g., ground chicken, chicken breast, check legs, chicken thighs, chicken wings, chicken patties, chicken tenders, chicken nuggets, chicken sausage, and the like. In some aspects, the meat substitute can mimic a turkey product, e.g., ground turkey, turkey sausage, turkey patties, and the like. In some aspects, the meat substitute can mimic a shellfish product, e.g., crab, lobster, shrimp, crayfish, clams, scallops, oysters, mussels, and the like. In some aspects, the meat substitute can mimic a cured, salted, or processed meat product, e.g., charcuterie, salami, summer sausage, prosciutto, bologna, kielbasa, and the like.

As used herein, the term “non-meat protein” refers to protein sourced from plants, fungus, insects, dairy products, or in vitro cultured animal cells, and excludes in vivo vertebrate animal derived tissues, cells, or proteins. For example, non-meat proteins may include plant-based proteins, fungal-based proteins, insect proteins, milk proteins (e.g., casein and whey), proteins from in vitro cultured animal cells, or combinations thereof.

As used herein, the terms “red chromogenic protein” (“RCP”) and “pink chromogenic protein” (“PCP”) are used interchangeably and refer to polypeptides which, when correctly folded and, if necessary, in the presence of required co-factors, have an absorbance spectrum maximum between 450 nm and 600 nm. The absorbance spectrum maximum is also referred to in the art as a lambda max. When in an aqueous solution at a concentration of at least 0.5 mg/ml, an RCP or PCP appears red or pink when viewed by the naked eye. RCPs and PCPs may also be referred to in the art as “red fluorescent proteins” and “pink fluorescent proteins.” PCP polypeptides described include PCPs from Pleurotus sp., for example PsPCP.

The PCP polypeptides described herein are characterized by a pink/red color and absorbance maximum between 450 nm and 600 nm when complexed with indol-3-one. The structure of indol-3-one is included below (formula I). For example, the PsPCP polypeptide alone is colorless and the indol-3-one compound alone is yellow with an absorption maximum at 456 nm. Without being bound to any particular theory, embodiment, or mode of action, the indol-3-one undergoes a bathochromic shift upon binding to the PsPCP polypeptide resulting in a PsPCP indol-3-one bound complex with an absorption maximum at 496 nm. The terms “indol-3-one bound PCP” and “PCP-indol-3-one complex” are used interchangeably herein and refer to complex formed between the PCP polypeptide and indol-3-one that results in the pink chromogenic complex with an absorption maximum at or around 496 nm.

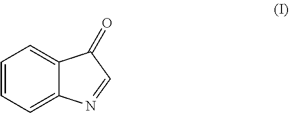

As used herein, the terms “polypeptide” and “peptide” are used interchangeably and refer to the collective primary, secondary, tertiary, and quaternary amino acid sequence and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, “PsPCP” refers to the PCP from the pink oyster mushroom Pleurotus salmoneostramineus. GenBank ID BBB05257.1. The wild-type polypeptide sequence of PsPCP is provided in SEQ ID NO:1.

SEQ ID NO: 1
MSLTLSPLPPLSNDIYPIGRNSLGNLMTATEKAKELPQEDKSAAQFQAT
SQESYKSAVSQTTKESPSASLAKFCKEAETAYPALYKAIQANDSASAKE
LAKSIASKLTEVATSAGNVAQAYNQGAAKAQEGQKLMKSALPGSHPVKD
SVDDALQYLSPAAQVFTSMQSSLNESAKNVVAAADKVGKVPANQIASED
SGEAIANAWAKLGVKATAQAEAYNKWQGNQ

As used herein, the terms “thermolabile RCP” and “thermolabile PCP” refer to an RCP or PCP polypeptide that, when heated at 130° C. for 2 minutes, has a decrease in absorbance at 496 nm relative to the absorbance at 496 nm prior to heating. In some aspects, after heating the thermolabile RCP or PCP has an absorbance of less than 80%, less than 70%, less than 60%, less than 50%, less than 40%, less than 30%, or less than 20% of the absorbance at 496 nm prior to heating. Visually, the intensity of the red or pink color of the thermolabile RCP or PCP may be reduced upon heating or the red or pink color may be completely absent following heating. Thermolabile RCPs and PCPs may be wild-type, naturally occurring RCPs and PCPs that show decreased red/pink color and absorbance at 496 nm upon heating. Thermolabile PCPs suitable for use in the pigments described herein include the thermolabile PsPCP of SEQ ID NO:1 and thermolabile PCP polypeptides at least 80%, at least 85%, at least 90%, at least 95%, or at least 98% identical to SEQ ID NO:1.

Variants or sequences having substantial identity or homology with the polypeptides described herein can be utilized in the practice of the disclosed pigments, compositions, and methods. Such sequences can be referred to as variants or modified sequences. That is, a polypeptide sequence can be modified yet still retain the ability to exhibit the desired activity. Generally, the variant or modified sequence may include or greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. There phrases also refer to DNA or RNA of natural or synthetic origin, which may be single-stranded or double-stranded and may represent the sense or the antisense strand. The DNA polynucleotides may be a cDNA or a genomic DNA sequence.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (i.e., polynucleotides encoding an EforRed polypeptide) may be codon-optimized for expression in a particular cell including, without limitation, a plant cell, bacterial cell, fungal cell, or animal cell. While polypeptides encoded by polynucleotide sequences found in coral are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

Also provided herein are polynucleotides encoding a thermolabile PsPCP polypeptide. The polynucleotide may encode any of the thermolabile PCP polypeptides described herein, for example, the polynucleotide may encode a polypeptide at least 80%, at least 85%, at least 90%, at least 95%, or at least 98% identical to SEQ ID NO:1.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding the thermolabile EforRed polypeptide. As used herein, the term “vector” refers to a polynucleotide capable of transporting another polynucleotide to which it has been linked. The vector may be a plasmid, which refers to a circular double-stranded DNA loop into which additional DNA segments may be integrated.

Cells including any of the polynucleotides, constructs, or vectors described herein are also provided. The cell may be a procaryotic cell or a eukaryotic cell. Suitable procaryotic cells include bacteria cell, for example, Escherichia coli and Bacillus subtilis cells. Suitable eukaryotic cells include, but are not limited to, fungal cells, plant cells, and animal cells. Suitable fungal cells include, but are not limited to, Fusarium venenatum, Pichia pastoris, Saccharomyces cerevisiae, Kluyveromyces lactis, Yarrowia lipolytica, Trichomderma reesei, Issatchenkia orientalis, and Aspergillus niger cells. Suitable plant cells include, but are not limited to, a pea cell (Pisum sativum), a corn cell (Zea mays), a soybean cell (Glycine max), and a wheat cell (Triticum sp.). Suitable animal cells include, but are not limited to, muscle cells (e.g., myocytes, myoblasts, myosatellite, and satellite cells) and fat cells (e.g., adipocytes or adipocyte progenitor cells such as mesenchymal stem cells). Suitable animal cells may be mammalian (e.g., bovine, porcine, and ovine), avian (e.g., poultry), crustacean (e.g., shrimp, lobster, and crab), mollusk (e.g., clam, mussel, scallop, and oyster) or insect cells. In some aspects, the cell is an edible mushroom cell, which refers to a mushroom that is safe for human consumption. For example, the edible mushroom cell can be a Fusarium venenatum, Agaricus bisporus, Lentinula edodes, or Volvariella volvacea cell.

Cells described herein may include indol-3-one and a polynucleotide, construct, or vector encoding a polypeptide at least 80%, at least 85%, at least 90%, at least 95%, or at least 98% identical to SEQ ID NO: 1. The indol-3-one may be introduced to the cell as an isolated compound, as a crude pink oyster mushroom extract, or may be produced from a biosynthetic pathway engineered into the cell. For example, the cell may include an exogenous polynucleotide encoding a dehydrogenase or reductase catalyzing the dehydrogenation of indoxyl to indolone. (FIG. 20). The cell may also include an exogenous polynucleotide encoding a cytochrome P450 monooxygenase catalyzing the formation of indoxyl from indole.

A recombinant host cell described herein and capable of producing a pink/red PsPCP-indol-3-one complex may include (i) an exogenous polynucleotide encoding a polypeptide at least 80%, at least 85%, at least 90%, at least 95%, or at least 98% identical to SEQ ID NO:1; (ii) a polynucleotide encoding a tryptophan feedback-insensitive 3-deoxy-D-arobino-heptulosonate 7-phosphate (DAHP) synthase; (iii) overexpression of the genes comprising the tryptophan biosynthetic pathway; (iv) an exogenous polynucleotide encoding the CYP102A cytochrome P450 monooxygenase from Streptomyces cattleya; and (v) an exogenous polynucleotide encoding an indoxyl dehydrogenase or reductase. In some aspects, the polynucleotide encoding the tryptophan feedback-insensitive DAHP is integrated into the genome of the host cell such that the native aroH DAHP synthase gene is replaced. The tryptophan biosynthetic pathway genes may be overexpressed by any suitable means known in the art, for example, introduction of a constitutive promoter, introduction of additional copies of the gene, and inhibiting repression of said gene expression such that the expression is increased. The recombinant host cell may be a bacterial, fungal, plant, or animal cell. In some aspects, the cell is an Escherichia coli, Bacillus subtilis, Fusarium venenatum, Pichia pastoris, Saccharomyces cerevisiae, Kluyveromyces lactis, Yarrowia lipolytica, Trichomderma reesei, Issatchenkia orientalis, Aspergillus niger, Agaricus bisporus, Lentinula edodes, Volvariella volvacea, Pisum sativum, Zea mays, Glycine max or Triticum sp. cell. In some aspects, the cell is an Escherichia coli, Bacillus subtilis, Fusarium venenatum, Pichia pastoris, Saccharomyces cerevisiae, Kluyveromyces lactis, Yarrowia lipolytica, Trichomderma reesei, Issatchenkia orientalis, or Aspergillus niger cell.

As used herein, feedback sensitivity refers to the inhibition of an enzyme, or inhibition of expression of an enzyme, by the reaction product of said enzyme or a product of the pathway in which the enzyme is active. For example, native fungal aroH DAHP enzymes are active in the tryptophan biosynthetic pathway but are inhibited by tryptophan feedback. The higher the concentration of tryptophan, the less DAHP enzyme activity. In contrast, expression of a feedback-insensitive DAHP enzyme results in consistent enzymatic activity even in the presence of high tryptophan concentration. Suitable tryptophan feedback-insensitive DAHP enzymes are known and described in the art. See, for example, Niu et al. (Niu, H, et al. Metabolic engineering for improving L-tryptophan production in Escherichia coli. J Indust Microbiol Biotechnol 46:55-65 (2019)) and Ger et al. (Ger et al. A single Ser-180 mutation desensitizes feedback inhibition of the phenylalanine-sensitive 3-deoxy-D-arabino-heptulosonae-7-phosphate (DAHP) synthase in Escherichia coli. J Biochem 116:989-990 (1994)). One skilled in the art will recognize tryptophan feedback-insensitive DAHP enzymes suitable for use as described herein.

A recombinant host cell described herein and capable of producing a pink/red PsPCP-indol-3-one complex may include (i) an exogenous polynucleotide encoding a polypeptide at least 80%, at least 85%, at least 90%, at least 95%, or at least 98% identical to SEQ ID NO:1; (ii) an exogenous polynucleotide encoding a tryptophanase; (iii) an exogenous polynucleotide encoding a CYP102A cytochrome P450 monooxygenase from Streptomyces cattleya; and (iv) an exogenous polynucleotide encoding an indoxyl dehydrogenase or reductase. The recombinant host cell may be a bacterial, fungal, plant, or animal cell. In some aspects, the cell is an Escherichia coli, Bacillus subtilis, Fusarium venenatum, Pichia pastoris, Saccharomyces cerevisiae, Kluyveromyces lactis, Yarrowia lipolytica, Trichomderma reesei, Issatchenkia orientalis, Aspergillus niger, Agaricus bisporus, Lentinula edodes, Volvariella volvacea, Pisum sativum, Zea mays, Glycine max or Triticum sp. cell. In some aspects, the cell is an Escherichia coli, Bacillus subtilis, Fusarium venenatum, Pichia pastoris, Saccharomyces cerevisiae, Kluyveromyces lactis, Yarrowia lipolytica, Trichomderma reesei, Issatchenkia orientalis, or Aspergillus niger cell.

Described herein are pigment compositions containing a thermolabile PsPCP, and meat substitutes including such pigment compositions. The pigment compositions disclosed herein can be used to provide color to a meat substitute that is similar to the color of natural animal meat when raw. Further, these pigment compositions change color upon heating and can provide an overall color change to the entire meat substitute composition that mimics the effects of cooking on natural animal meat. In an aspect, the pigment composition provides a pink and/or red color to raw, uncooked meat substitute that transitions to a brown, white, colorless, or less red color after cooking the meat substitute.

The pigment composition itself loses its pink or red color as it is cooked due to degradation and may become colorless if enough degradation occurs. Accordingly, the brown color of a cooked meat substitute is not necessarily due to the pigment composition turning brown in color, but instead due to the pigment composition losing its reddish color. The degraded pigment composition in the cooked meat substitute no longer masks the other colors of the meat substitute and the brown colors associated with Maillard reactions in the meat substitute become more apparent.

The redness or pinkness of the pigment composition is reduced substantially or eliminated when heated to a temperature within a range typically used for cooking meat. The pigment composition changes from a pink and/or red color to a less-pink/red color or becomes substantially colorless when heated at 80° C. for 20 minutes. The pigment composition can be used to change the color of a meat substitute from a pink and/or red color to a brown color and/or less pink/red color, as exhibited by heating a meat substitute including the pigment composition at 80° C. for 20 minutes.

The changes in color of a pigment composition sample can be measured using a Hunter Colorimeter and reported as a relative percent change in visible light absorbance after heating as compared to the sample prior to heating. When the thermolabile PsPCP, the pigment composition, or the meat substitute is heated on a hot plate at 130° C. for 90 seconds, the a* value of L*a*b* colorimetry decreases relative to the a* value prior to heating. The a* value may decrease by at least 5%, at least 10%, at least 15%, at least 20%, at least 25%, at least 30%, at least 35%, at least 40%, at least 45%, or at least 50%. Likewise, when the thermolabile PsPCP, the pigment composition, or the meat substitute is heated at 80° C. for 20 minutes the absorbance of light at a wavelength of 496 nm is decreased relative to the absorbance prior to heating. The absorbance at 496 nm may decrease by at least 5%, at least 10%, at least 15%, at least 20%, at least 25%, at least 30%, at least 35%, at least 40%, at least 45%, or at least 50%.

The pigment compositions described herein include a thermolabile PsPCP. For example, the pigment composition may include a polypeptide with a sequence at least 80%, at least 85%, at least 90%, at least 95%, or at least 98% identical to SEQ ID NO:1. The pigment composition may include an aqueous pink oyster mushroom extract (e.g., extract of Pleurotus djamor or Pleurotus salmoneostramineus) comprising the thermolabile PsPCP. The pigment composition may include pink oyster mushroom.

The pigment composition additionally includes indol-3-one at a concentration suitable to for a complex with the PsPCP, said complex having an absorbance maximum of about 496 nm. The indol-3-one may be included in the pigment composition in a molar ratio between 0.5:1 to 2:1 with the PsPCP polypeptide. The indol-3-one may be included in the pigment composition in a weight ratio between 1:86 to 1:344 with the PsPCP polypeptide. In aspects in which the pigment includes a pink oyster mushroom extract or pink oyster mushroom, it is expected that both the PsPCP and the indol-3-one are included in the pigment in the correct ratio as both the extract and the pink oyster mushroom have the desired pink color as demonstrated herein.

The pigment composition can be included in a meat substitute at a level that provides increased or improved pink and/or red color in the meat substitute, while also providing increased or improved brown color in the meat substitute after cooking. In an aspect, the pigment composition is used at a level to provide at least 0.01%, 0.05%, 0.1%, 0.2%, 0.3%, 0.4%, 0.5%, 1.0%, 1.25%, or 1.5% of the PCP-indol-3-one complex on a wet (total) weight basis in a meat substitute composition. The pigment composition may be used at a level such that the PCP-indol-3-one complex is present in the meat substitute composition the range of 0.01% to 6%, 0.05% to 5%, 0.1% to 3%, or 0.5% to 2% by weight in a meat substitute composition.

The pigment composition may additionally include a carrier or a diluent. The PCP-indol-3-one complex may be added to a liquid composition used in producing the meat substitute composition, for example, a liquid brine composition. The PCP-indol-3-one complex may be included in the liquid (e.g., liquid brine) composition at any suitable concentration such that the meat substitute composition includes between 0.01%-6%, 0.05%-5%, 0.1%-3%, or 0.5%-2% of the PCP-indol-3-one complex by weight of the meat substitute composition. For example, the PCP-indol-3-one complex may be included in the liquid composition at a concentration between 0.1% and 60%, between 0.5% and 50%, between 1% and 40%, or between 2% and 30% by weight of the liquid composition.

The pigment composition may be a dry lyophilized powder that is or comprises the PCP-indol-3-one complex. The dry lyophilized powder may be added to a liquid composition (e.g., a liquid brine composition) prior to formation of the meat substitute composition or the dry lyophilized powder may be added to the meat substitute composition directly. The dry lyophilized powder is added to a liquid composition for the production of a meat substitute or added directly to the meat the substitute such that the resulting meat substitute composition between 0.01%-6%, 0.05%-5%, 0.1%-3%, or 0.5%-2% of the PCP-indol-3-one complex by weight of the meat substitute composition.

The pigment composition may also include a blend of the PsPCP polypeptide and indol-3-one with another color or pigment. For example, the pigment composition may include the PsPCP polypeptide and indol-3-one with a fruit or vegetable extract-based pigment composition.

The pigment composition described herein can be used as a pigment in any meat substitute composition. An exemplary, but non-limiting, meat substitute composition is a composition which comprises a combination of plant protein (e.g., textured pea protein and/or pea protein), water, vegetable oil, flavor ingredients, salts, sugars, binders, and the pigment composition described herein. The pigment composition described herein can also be used in food applications other than meat substitutes.

Meat substitutes described herein may include one or more cells comprising an exogenous polynucleotide encoding a thermolabile PsPCP as described herein. For example, the meat substitutes may include a fungal, plant, or animal cell as described herein comprising an exogenous polynucleotide encoding a thermolabile PsPCP polypeptide described herein. The meat substitutes may also include a fungal, plant, or animal cell as described herein comprising an exogenous polynucleotide encoding a thermolabile PsPCP polypeptide and one or more exogenous polynucleotides encoding one or more biosynthetic pathway enzymes for the production of indol-3-one.

The meat substitutes described herein may include a non-mean protein and a pigment composition comprising pink oyster mushroom. The pink oyster mushroom may be prepared in a form suitable for inclusion in the pigment or meat substitute composition including, but not limited to, chopped, ground, pureed, crushed, or dried. The meat substitutes may include a non-meat protein and an aqueous extract of pink oyster mushroom, including, but not limited to, an aqueous extract of Pleurotus djamor or Pleurotus salmoneostramineus.

Also provided herein is a method for increasing the red color of a meat substitute. The method for increasing the red color of a meat substitute includes adding a thermolabile indol-3-one bound PsPCP polypeptide to a meat substitute prior to cooking the meat substitute, wherein the red color of the meat substitute is increase relative to the meat substitute without the thermolabile indol-3-one bound PsPCP polypeptide. The method may also include adding a thermolabile indol-3-one bound PsPCP polypeptide to a non-meat protein to form a meat substitute with increased red color relative to the non-meat protein without the indol-3-one bound PsPCP polypeptide. The thermolabile indol-3-one bound PsPCP polypeptide may be any thermolabile PsPCP polypeptide as described herein. For example, the thermolabile RCP polypeptide to be added to the meat substitute may comprise a sequence at least 80%, at least 85%, at least 90%, at least 95%, or at least 98% identical to SEQ ID NO:1.

Also provided is a method for decreasing red color in a cooked meat substitute. The method for decreasing the red color in a cooked meat substitute includes cooking a meat substitute comprising a non-meat protein and a thermolabile indol-3-one bound PsPCP polypeptide, whereby red color of the cooked meat substitute is reduced relative to the meat substitute prior to cooking. The thermolabile indol-3-one bound PsPCP polypeptide may be any thermolabile PsPCP polypeptide as described herein. For example, the thermolabile RCP polypeptide to be added to the meat substitute may comprise a sequence at least 80%, at least 85%, at least 90%, at least 95%, or at least 98% identical to SEQ ID NO:1.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1: Pink Oyster Mushroom Extract

Pink oyster mushrooms (Pleurotus djamor) was purchased from R&R Cultivation. An aqueous extract of the pink oyster mushrooms was prepared by chopping the mushroom caps and stems, adding water to the chopped mushroom, and incubating overnight at 4° C. to extract the pink pigment. Pigment extraction was also attempted using ethanol, however addition of ethanol to the chopped mushroom resulting in discoloration. Further experiments were carried out with an aqueous extraction.

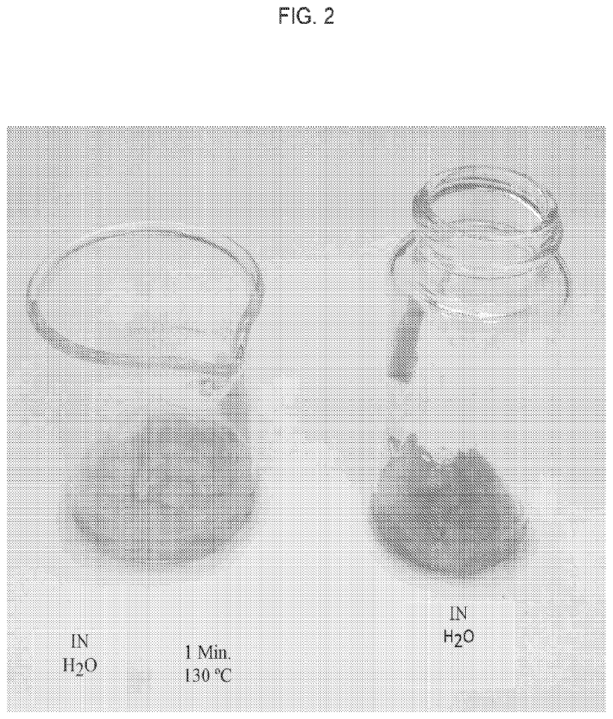

Heat stability of the chopped pink oyster mushroom steeped in water was observed by heating the extract on a 130° C. hot plate for 1 minutes. As shown in FIG. 2, prior to heating the chopped mushroom is pink in color (right), but after heating the pink color is lost (left).

### Example 2: Aqueous Pink Oyster Mushroom Extracts

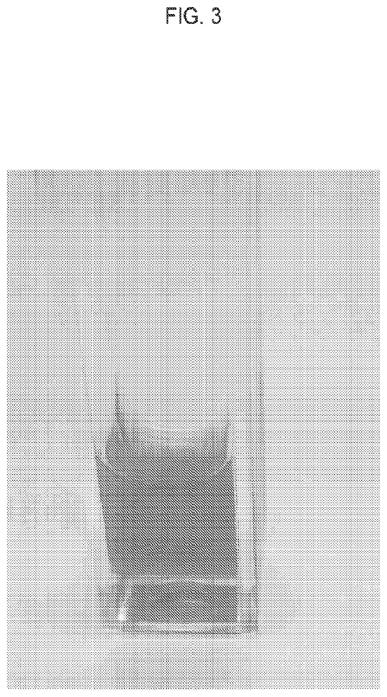

Pink oyster mushrooms (Pleurotus djamor) was purchased from R&R Cultivation. An aqueous extract of the pink oyster mushrooms was prepared by chopping the mushroom caps and stems, adding water to the chopped mushroom, and pureeing the suspension using a mortar and pestle. The puree was centrifuged, and a pink supernatant was collected. (FIG. 3)

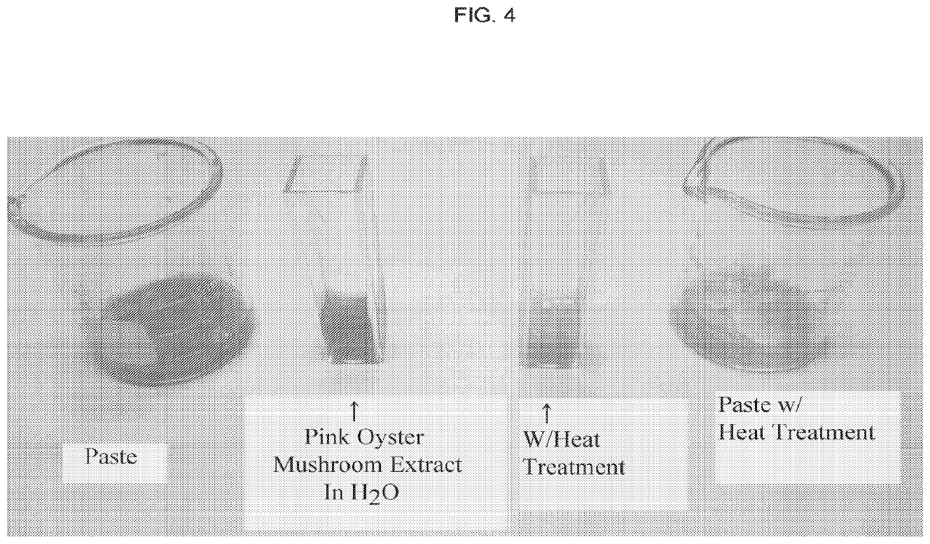

Heat stability of the aqueous extract was observed by heating the extract on a 130° C. hot plate. After heating the pink supernatant for 20 seconds, the pink color was lost. After an additional 20 seconds, protein coagulation occurred, and a precipitate was observed. Heat stability of the puree was also tested by heating on a 130° C. hot plate for 2 minutes. Pink color loss in the puree was observed after 50 seconds of heating. (FIG. 4) The paste remaining after centrifugation of the puree was also slightly pink (not shown) indicated incomplete extraction of the pink pigment component.

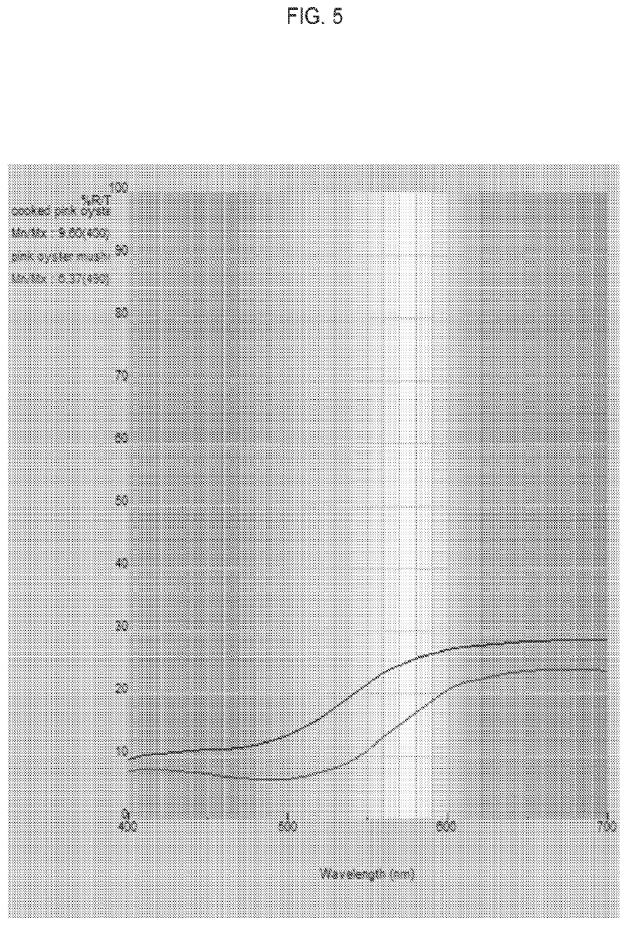

Hunter colorimetry data for the pureed pink oyster mushroom extract before and after heating (130° C. hot plate, 2 minutes) is reported in Table 2 and FIG. 5.

TABLE 2

| | L* | a* | b* | L* | C* | h |
|---|---|---|---|---|---|---|
| pink oyster mushroom puree raw | 42.57 | 18.94 | 17.48 | 42.57 | 25.77 | 42.7 |
| cooked pink oyster mushroom puree | 53 | 8.15 | 22.19 | 53 | 23.64 | 69.83 |

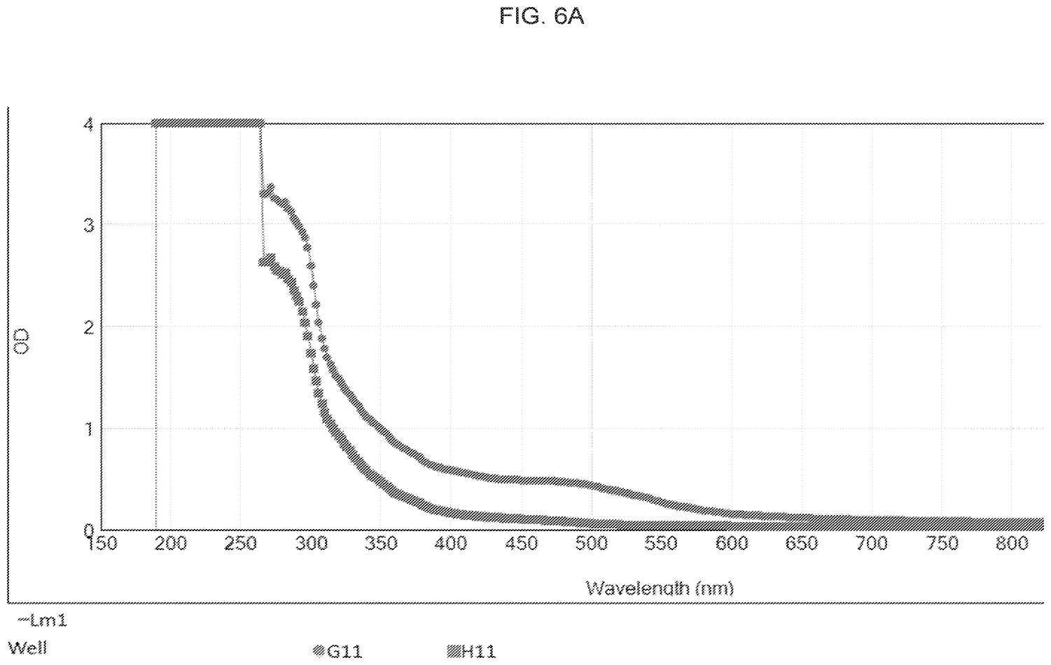

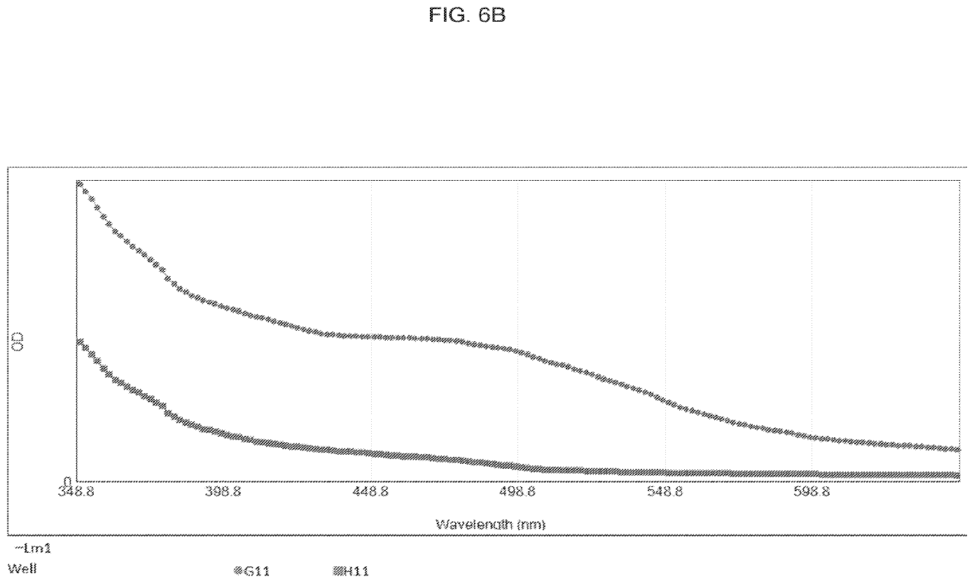

Absorbance spectrum of the aqueous extract supernatants were measured using a Spectramax plate reader. The heated sample was first centrifuged to remove any precipitate. The measured absorbance spectra before (“G11”) and after (“H11”) heating are reported in FIGS. 6A and 6B.

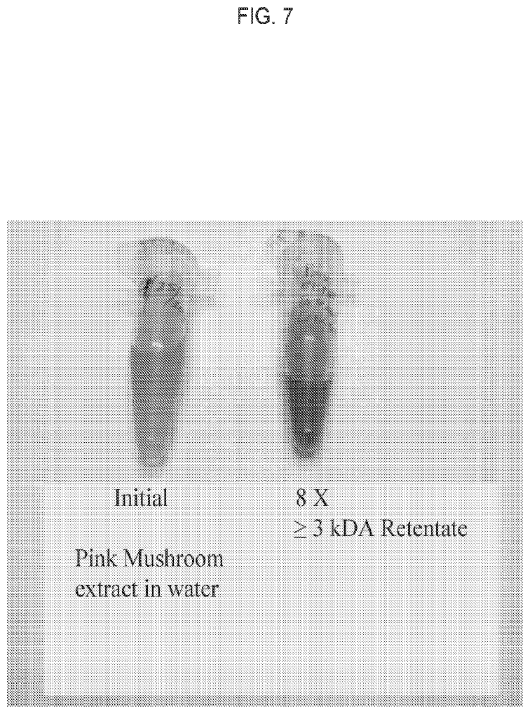

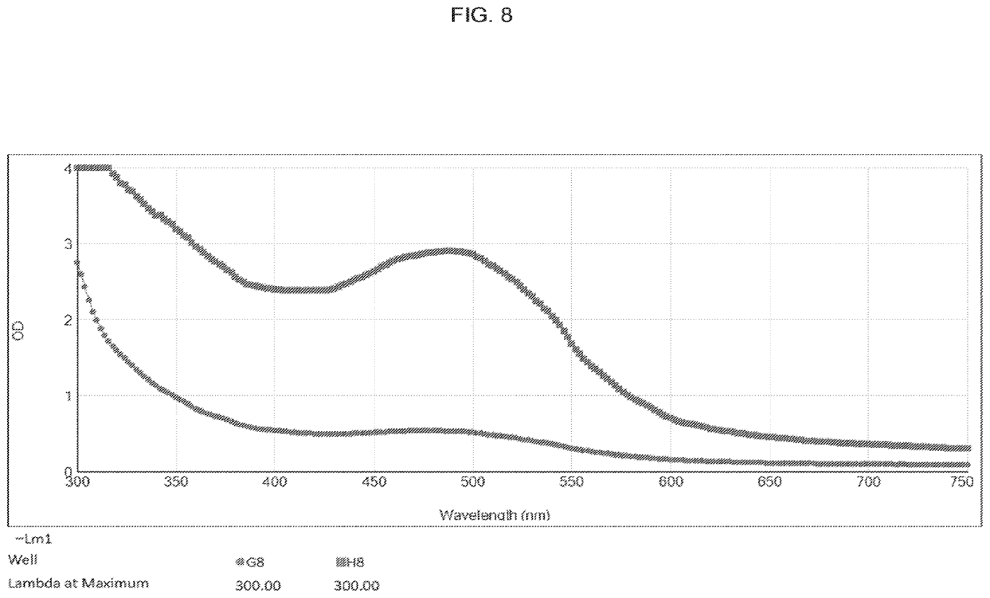

An 8× concentrate of the aqueous pink oyster mushroom extract was prepared by concentrating 4 ml (about 1.54 mg/ml) of the aqueous extract supernatant to 0.5 ml using a 3 kDA molecular weight cutoff filter. Protein concentration in the supernatant was determined using the Pierce 600 reagent against a BSA standard. The 8× concentrate had a more prominent red color then initial supernatant (FIG. 7) and the filtrate was yellow. The absorbance spectrum of the 8× concentrate demonstrates the characteristic absorbance peak of 440-540 nm that would be expected for a pink or red chromogenic protein (FIG. 8), with an absorbance maximum of about 490 nm.

### Example 3: Pink Oyster Mushroom Puree Colorimetry

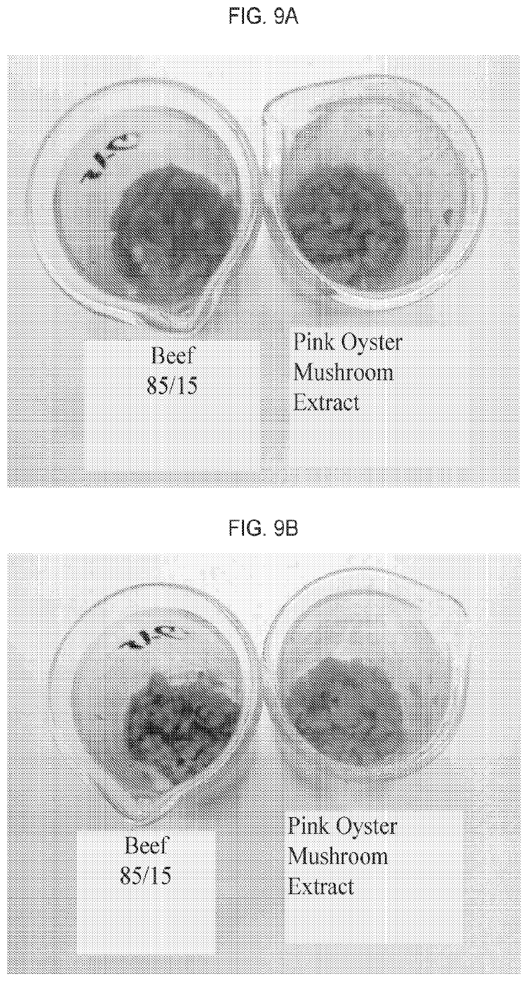

Hunter colorimetry data was collected for raw beef (85/15) and a meat substitute composition including the concentrated pink oyster mushroom extract and pea protein. Likewise, colorimetry data was collected for each sample, beef (85/15) and meat substitute, following cooking on a 130° C. hotplate for 1 minutes. Data are reported in Table 3. As demonstrated in FIG. 9A, the hue of the raw pink oyster mushroom puree is close to that of the raw beef. When cooked, the pink oyster mushroom puree turned pale (higher L* value) and the beef turned darker brown (decreased L* value) (FIG. 9B).

TABLE 3

| | Raw | | | | | | Cooked | | | | | |
|---|---|---|---|---|---|---|---|---|---|---|---|---|
| | L* | a* | b* | C* | h | dE* | L* | a* | b* | C* | h | dE* |
| beef 85/15 | 44.7 | 22.9 | 17.3 | 28.7 | 37.1 | | 39.4 | 5.6 | 14.7 | 15.8 | 69.1 | |
| pink oyster mushroom | 48.2 | 22.5 | 19.2 | 29.6 | 40.5 | 4.0 | 52.0 | 15.3 | 24.9 | 29.2 | 58.4 | 18.8 |

### Example 4: Source of Color in Pink Oyster Mushroom Extracts

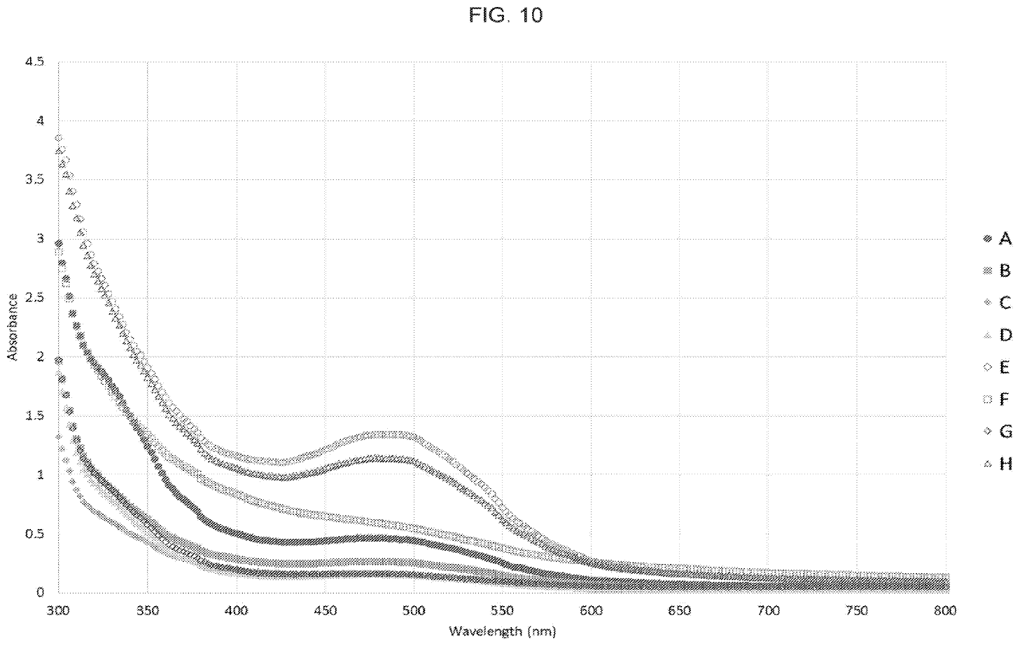

6 g of frozen (about 80° C.) pink oyster mushroom were pureed using a mortar and pestle and the addition of about 10 mL of water. The puree was vortexed and stored at 4° C. overnight. The puree was then centrifuged, and the supernatant collected (Fraction A). The remaining pellet was still pink (based on observation with the human eye) and two additional water extractions were carried out, collecting the supernatant from each collection as Fractions B (second extraction) and C (third extraction). Each fraction was stored at 4° C. Separate portions of Fraction A were concentrated by filtering through a 100,000 molecular weight cutoff (“MWCO”) filter to from Fractions D and E and a 50,000 MWCO filer for form Fractions G and H. Both the filtrate and the retentate fractions from each filtration were collected (Fractions E-F and G-H). A fraction (F) was also collected from a wash of the 100,000 MWCO filter following concentration. Fractions A-H are summarized in Table 4. FIG. 10 shows absorbance spectra collected for each of Fractions A-H.

TABLE 4

| | Net | Fraction | | |
|---|---|---|---|---|
| | Protein | volume | Total | |
| | conc. | collected | protein | |
| Fraction | (mg/mL) | (mL) | (mg) | Fraction description |
| A | 1.016 | 6.75 | 6.86 | Extract 1st fraction |
| B | 0.597 | 6.15 | 3.67 | Extract 2nd fraction |
| C | 0.296 | 5.2 | 1.54 | Extract 3rd fraction |
| D | 0.187 | 3.1 | 0.58 | Frac A: Filtrate of |
| | | | | 100,000 MWCO |
| E | 3.224 | 0.8 | 2.58 | Frac A: Retentate of |
| | | | | 100,000 MWCO |
| F | 0.900 | 0.2 | 0.18 | Frac A: Wash membrane |
| | | | | of 100,000 MWCO |
| G | 0.071 | 0.65 | 0.05 | Frac A: Filtrate of |
| | | | | 50,000 MWCO |
| H | 2.591 | 0.3 | 0.78 | Frac A: Retentate |
| | | | | of 50,000 MWCO |

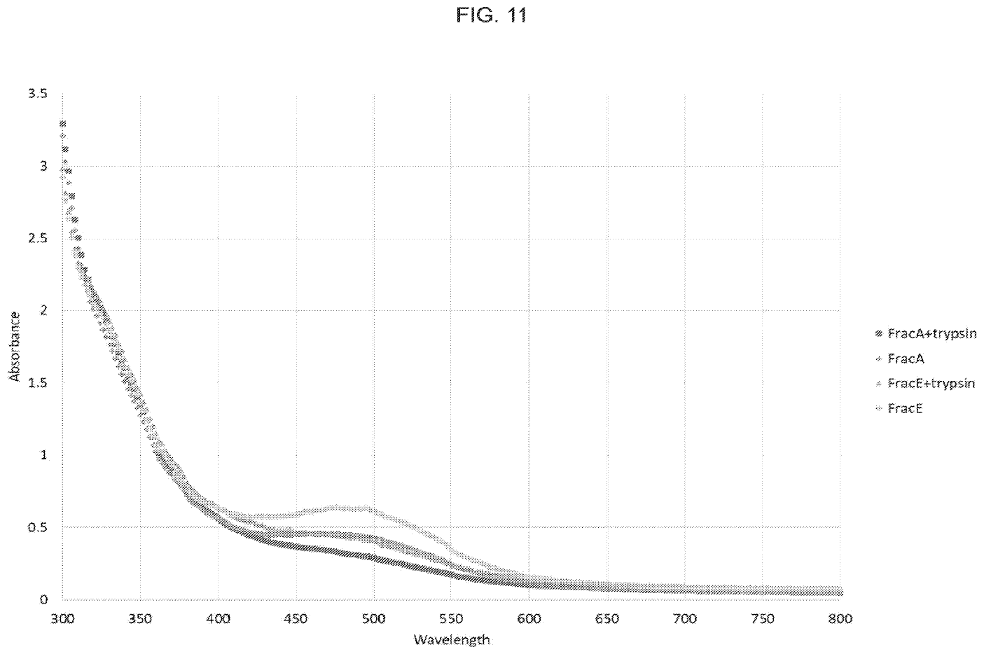

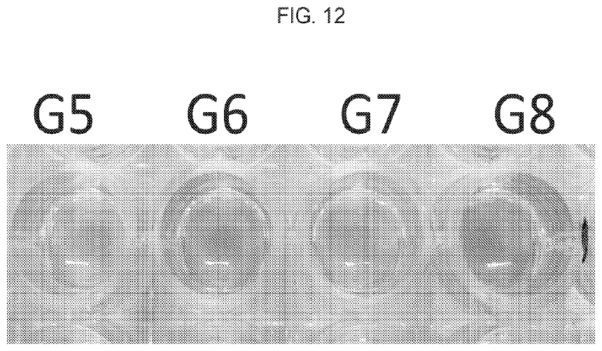

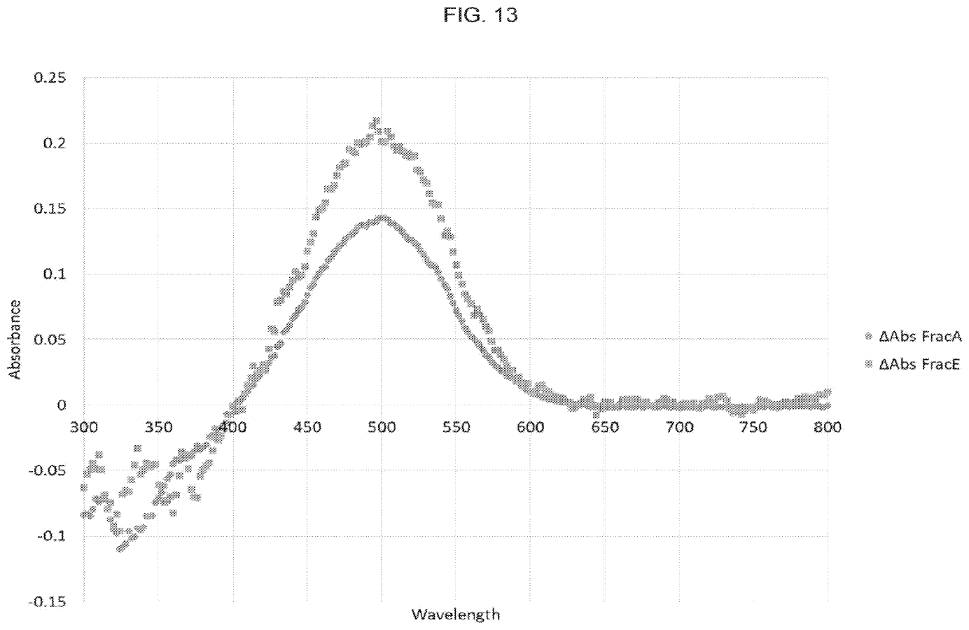

To determine if the colored component of the pink oyster mushroom was a protein, trypsin digest was carried out on Fractions A and E. Trypsin is a protease that specifically cleaves the peptide bond at the C-terminal end of lysine and arginine residues. Samples were included for 6 hours at room temperature under the conditions outlined in Table 5. A photo of Fractions A and E with and without trypsin digest is shown in FIG. 12, and the absorbance spectra are shown in FIG. 11. The change in absorbance between the undigested and digested samples is shown in FIG. 13. The colored component of the pink oyster mushroom is a protein, as the pink color and absorbance peak between 440 and 540 nm are both reduced upon trypsin digest.

TABLE 5

| Sample (6 h reaction at RT) | Well |
|---|---|
| Frac A 100 uL + 10 uL Trypsin Lys-C | G5 |
| Frac A 100 uL + 10 uL water | G6 |
| Frac E (1:2 diluted in 50 mM HEPES) | G7 |
| 100 uL + 10 uL Trypsin Lys-C | |
| Frac E (1:2 diluted in 50 mM HEPES) | G8 |
| 100 uL + 10 uL water | |

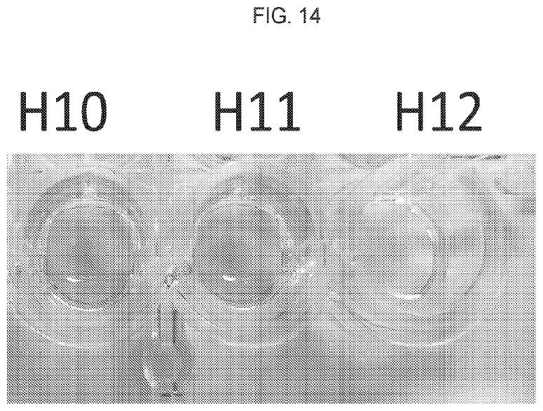

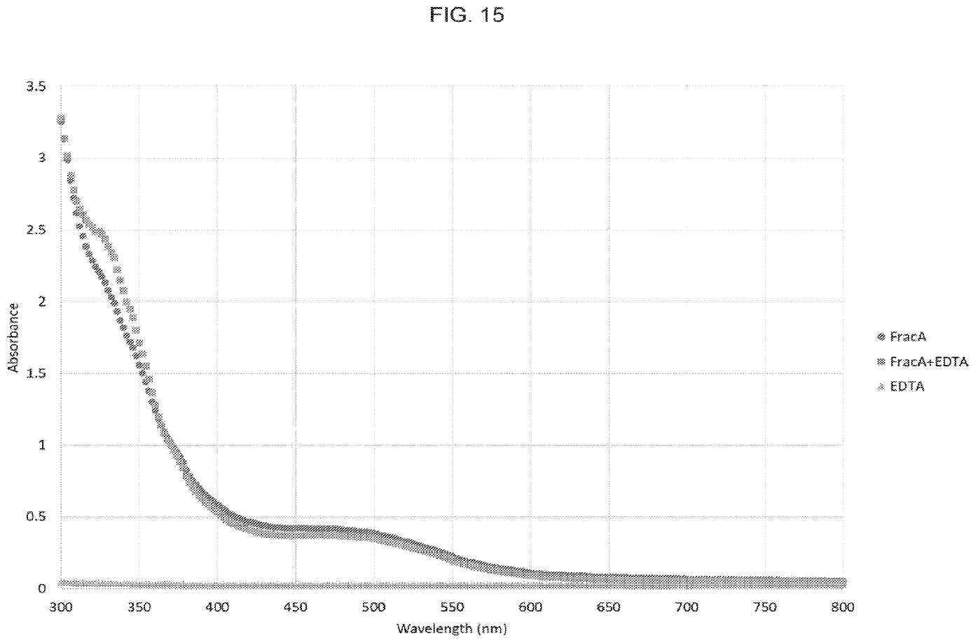

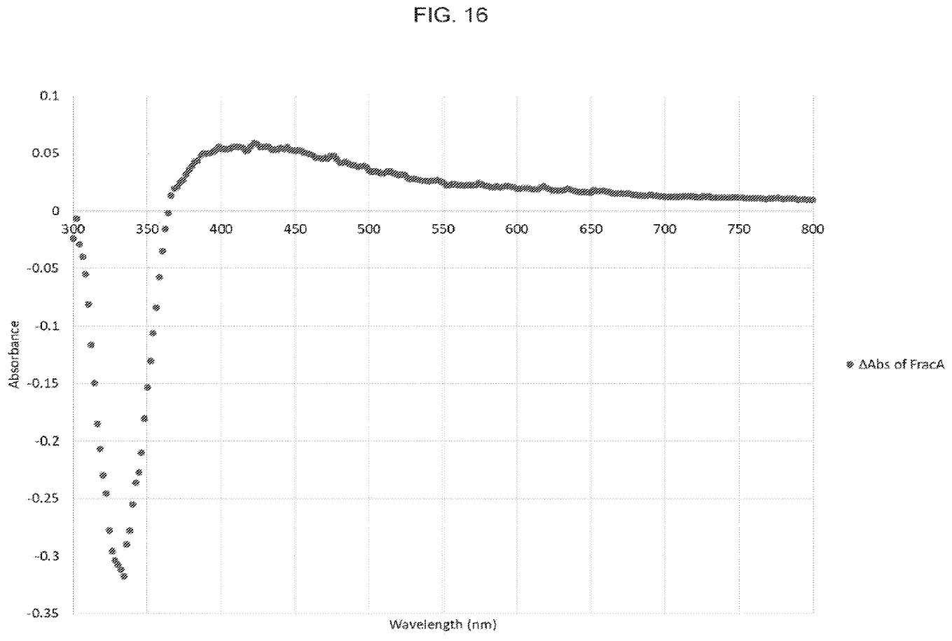

To determine if the colored component of the pink oyster mushroom requires metals ions, ethylenediaminetetraacetic acid (EDTA) treatment was carried out on Fraction A. EDTA is a metal chelating agent and strips metal ions from proteins. Samples were incubated overnight in the conditions outlined in Table 6. A photo of Fraction A with and without EDTA treatment is shown in FIG. 14 and includes an EDTA alone sample for comparison. The absorbance spectra are shown in FIG. 15. The change in absorbance between the EDTA treated and untreated samples is shown in FIG. 16. The EDTA treatment did not reduce the pink appearance or the absorbance peak between 440 and 540, so it is likely that a metal ion is not required for the pink color of the protein in the pink oyster mushroom extracts.

TABLE 6

| Sample (overnight treatment at RT) | Well |
|---|---|
| Fraction A + water | H10 |
| Frac A + EDTA (90 mM final) | H11 |
| 50 mM EDTA in water | H12 |

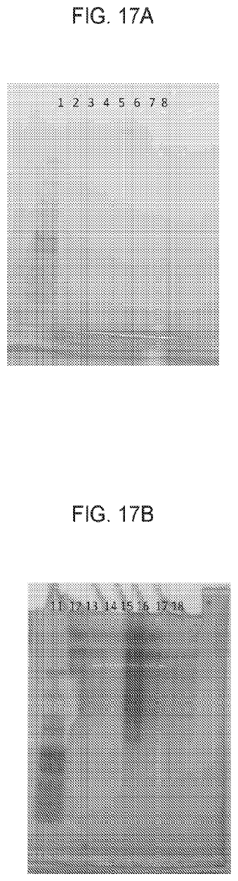

Native (non-denaturing) gel electrophoresis was used to isolate the colored protein for proteomic analysis. FIG. 17A show the unstained native gel, in which a faint pink band is visible in lanes 5 and 6. FIG. 17B shows a stained native gel, in which the band corresponding to the colored protein is visible in lanes 15 and 16. The content of each of the lanes of the gel is outlined in Table 7. The bands from lanes 5, 6, 15, and 16 that correspond to the pink colored protein were isolated for proteomic analysis.

TABLE 7
Native gel electrophoresis

| | Unstained | Stained | Amount |
|---|---|---|---|
| | Well | Well | of protein |
| Fraction | (FIG. 17A) | (FIG. 17B) | loaded (μg) |
| A | 2 | 12 | 10 |
| B | 3 | 13 | 5.9 |
| D (Filtrate) | 4 | 14 | 1.9 |
| E (Retentate 100,000) | 5 | 15 | 32.2 |
| H (Retentate 50,000) | 6 | 16 | 10 |
| Frac A + trypsin (37 C.) | 7 | 17 | 10 |
| Frac A (37 C.) | 8 | 18 | 10 |

### Example 5: Proteomics

The bands from lanes 5, 6, 15, and 15 of the native gel electrophoresis run in Example 4 (FIGS. 17A and 17B) were excised and prepared for proteomics analysis via liquid chromatography/mass spectrometry (LC/MS). Post excision, the protein bands were processed for bottom-up proteomics analysis with the Thermo Scientific In-gel Tryptic Digestion Kit (catalog #89871). The kit includes materials for protein extraction from the gel, denaturation, alkylation, and trypsin digestion. Trypsin digested samples were injected onto a high performance liquid chromatography (HPLC) column (Acclaim Vanquish C18 Column, 250×2.1, 2.2 um part #0748125-V) using a Thermo Vanquish autosampler. The HPLC column was coupled to a Thermo Fusion Lumos mass spectrometer set to MIPS (monoisotopic precursor selection) peptide mode at 120,000 resolution. Data analysis was performed via Thermo Proteome Discoverer software.

Based on previous experience with indole moiety binding proteins, a literature search was preformed for evidence of similar pink chromogenic proteins. The search identified work by Fukuta et al. (“Gene cloning of the pink-colored protein from Pleurotus salmoneostramineus (PsPCP) and its species-specific chromoprotein are effective for colorization of the fruit body,” Biosci Biotechnol Biochem 83:1354-1361 (2019)) identifying a pink chromogenic protein from Pleurotus salmoneostramineus (PsPCP). The LC/MS peptide data gathered from the excised gel bands was compared to the amino acid sequence of the PsPCP reported by Fukuta, and a greater than 90% sequence match was identified. While Fukuta reported the PsPCP, this is believed to be the first identification of the PCP in Pleurotus djamo.

### Example 6: Recombinant PsPCP Expression

The pink chromogenic protein (PsPCP) from pink oyster mushroom Pleurotus salmoneostramineus comprises the polypeptide of SEQ ID NO: 1 (Fukuta et al., Biosci Biotechnol Biochem 83:1354-1361 (2019)) and an associated pigment identified as indolone (3H-indol-3-one, Takekuma et al., J Am ChemSoc 116:8849-8850 (1994)). The polypeptide by itself is colorless and indol-3-one by itself is yellow (absorption maximum at 456 nm), However, the indol-3-one undergoes a bathochromic shift upon binding to the PSP polypeptide. As extracted from the mushrooms, the polypeptide-indolone complex is pink/red in color with an absorption maximum at 496 nm.

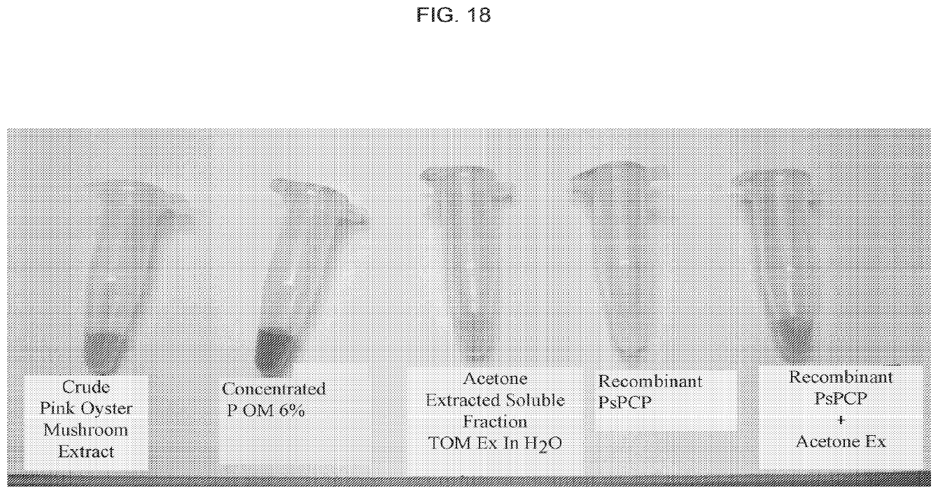

A synthetic gene encoding the His6-tagged PsPCP polypeptide was obtained (GENEWIZ, NJ, USA) and expressed in E. coli BL21 (DE3) using the T7 promoter/polymerase system. The sequence of the His6 tag was MGSSHHHHHHSSGLVPRGSH (SEQ ID NO:4). PsPCP constituted 22% of the soluble protein in lysates, and was readily purified using NiNTA (HisPur™, ThermoFisher Scientific, Waltham, MA) batch chromatography. The expressed protein was purified using Ni-NTA affinity binding and was observed to be colorless. (FIG. 18)

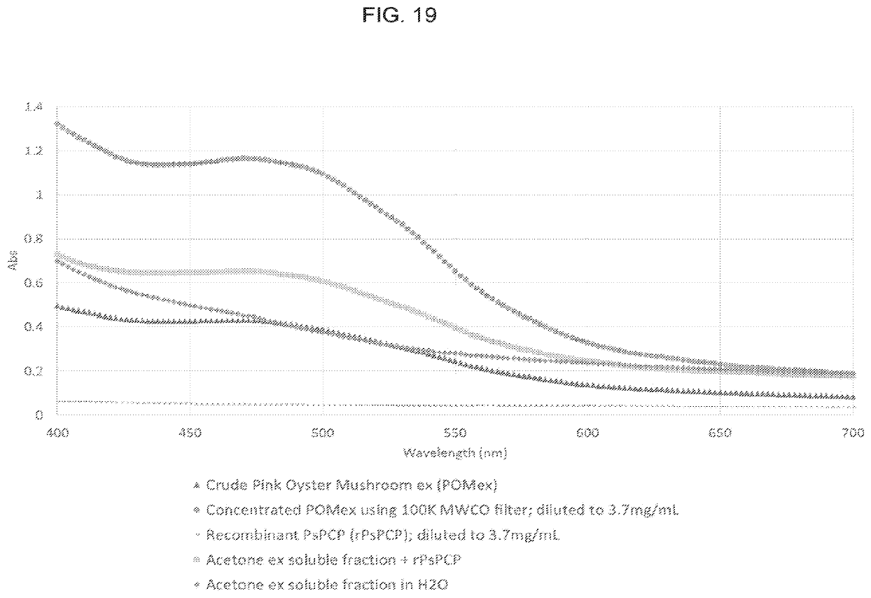

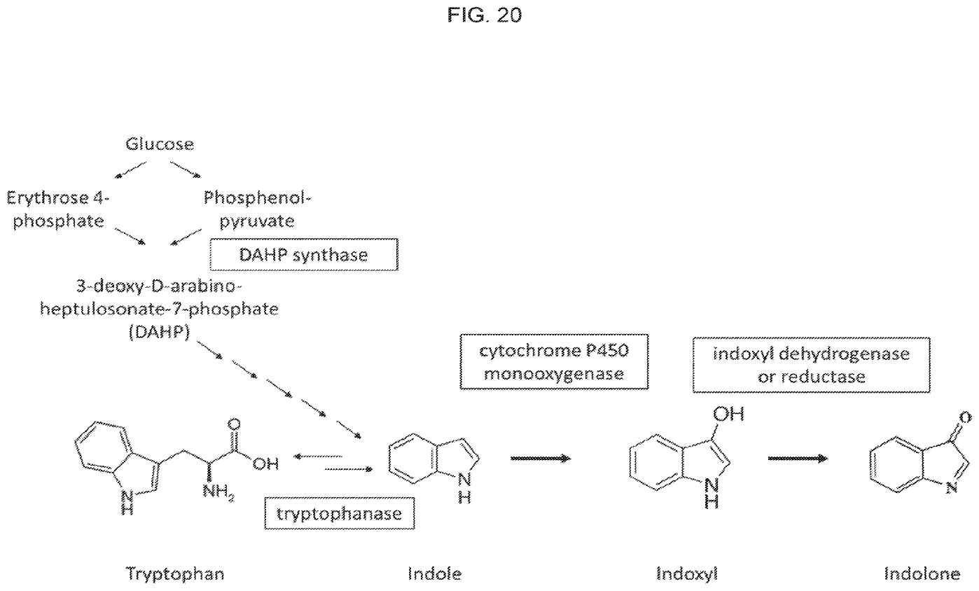

Indol-3-one, the small molecule needed for the colored complex generation, was extracted from the pink oyster mushroom in a crude extract using acetone. This extract, when mixed with the colorless purified PsPCP, resulted in a pink pigment and the absorption spectra observed was similar to the crude pink oyster mushroom extract. (FIG. 19)

Acetone extraction of a small molecule fraction containing the indol-3-one molecule was carried out using the following steps:
1. Crush the pink oyster mushroom and extract the water-soluble fraction (POMex)
2. Concentrate the water-soluble extract using a 100 kDa molecular weight cut off filter
3. Add acetone to the retentate to a final concentration of 60%
4. Incubate the acetone retentate mixture at 4 C for 30 minutes
5. Centrifuge at 14,000 g to pellet the precipitate
6. Collect supernatant containing the acetone faction including small molecules (e.g., indol-3-one)
7. Dry the acetone fraction under nitrogen to produce a small molecule powder containing indol-3-one

### Example 7: Recombinant Expression of PsPCP and Indol-3-One

A recombinant host cell that produces indol-3-one while expressing the PsPCP gene would generate the pink/red colored complex directly. The proposed metabolic pathway for the production of indol-3-one is shown in FIG. 20. The immediate precursor for indol-3-one is proposed to be indoxyl (3-hydroxy-indole). Conversion of indoxyl to indol-3-one can be carried out enzymatically by a dehydrogenase or reductase. There are many dehydrogenases and reductases known in the art, and methods to screen genes for said enzymatic activity are well known. One such dehydrogenase is the polyol dehydrogenase PDH-11300 from Deinococcus geothermalis (SEQ ID NO:2, GenBank ABA78522) (Wulf et al., Enz Microbioal Technol 51:217 (2012)) which has been shown to have broad substrate specificity. Alternatively, the gene encoding the specific dehydrogenase or reductase may be isolated from the genome of the pink oyster mushrooms by well-known gene cloning methods.

SEQ ID NO: 2
MDYRTVFRLDGACAAVTGAGSGIGLEICRAFAASGARLILIDREGAALD
RAAEELGAAVASRIVADVTDAEAMTAAAAAAEAVAPVSILVNSAGIARL
HDALETDDATWRQVMAVNVDGMFWASRAFGRAMVARGAGAIVNLGSMSG
TIVNRPQFASSYMASKGAVHQLTRALAAEWAGRGVRVNALAPGYVATEM
TLKMRERPELFGTWLDMTPMGRCGEPSEIAAAALFLASPAASYVTGAIL
AVDGGYTVW

Indoxyl is generated from indole as the product of the reaction catalyzed by numerous classes of enzymes including naphthalene dioxygenases, multicomponent phenol hydroxylases, cytochrome P450 monooxygenases, peroxygenases, and flavin-dependent monooxygenases such as indole monooxygenases (Fabara and Fraaije, Appl Microbiol Biotechnol 104:925-933 (2020)). The pathways to the production of indoxyl have been investigated extensively as indoxyl is also the precursor to indigo, a natural blue dye. Expression of the CYP102A cytochrome P450 monooxygenase from Streptomyces cattleya (SEQ ID NO:3, GenBank CCB77526) has been found to be sufficient to produce indigo (Kim et al., Dyes and Pigments 140:29-35 (2017)), implying that it produces the indoxyl precursor.

SEQ ID NO: 3
MSPTPHSASGTTGAAAATPGAASPAPPVPVADISDTGFGTTPIQQAMAL
AREHGPVFRRRFGTFESLLVGSVDAVTELCDDERFVKAVGPVLTNVRQI
AGDGLFTAYNDEPNWAKAHDILLPAFALSSMHTYHPTMLRVAKRLIAAW
DTALADGAPVDVADDMTRMTLDTIGLAGFGYDFGSFRRGEPHPFVAAMV
RGLLHSQALLSRKADDGVDHSAADEAFRADNAYLAQVVDEVIEARRASG
ETGTDDLLGLMLGAPHPSDGTPLDAANIRNQVITFLIAGHETTSGALSF
ALYYLAKNPAVLRRAQAEVDALWGDDPDPEPDYTDVGRLTYVRQVLNEA
LRLWPTAAAFGRQAVTDTVLDGRVPMRAGDTALVLTPVLHRDPVWGDNV
EAFDPERFSPEREAARPVHAFKPFGTGERACIGRQFALHEAVMLLGMLI
HRYRFLDHADYRLRVRETLTLKPDGFTLKLARRTSADRVRTVASRAAEG
TAGQDAGLPTTARPGTTLTVLHGSNLGACREFAAGLADLGERCGFETTV
APLDAYRAGDLPRTSPVVVVAASYNGRPTDDAAGFVSWLEQAGPGAADG
VRYAVLGVGDRNWAATYQKVPTLIDERLAECGATRLLERAAADAAGDLA
GTVRGFGEALRRALLAEYGDPDSVGAVAGAEDGYEVTEVTGGPLDALAA
RHEVVAMTVTETGDLADLTHPLGRSKRFVRLALPDGATYRTGDHLAVLP
ANDPALVERAARLLGADPDTVLGVRARRPGRGTLPVDRPVTVRELLTYH
LELSDPATAAQIAVLADRNPCPPEQAELKKLAPGRASVLDLVERYPALT
GRLDWPTVLGTLLPQIRIRHYSVSSSPAVSPGHVDLMVSLLEADGRRGT
GSGHLHRVRPGDVVYARVAPCREAFRIAAGDEVPVVMVAAGTGLAPFRG
AVADRVALRSAGRELAPALLYFGCDHPEVDFLHAAELRGAEAAGAVSLR
PAFSAAPDGDVRFVQHRIAAEADEVWSLLKGGARVYVCGDGSRMAPGVR
EAFTALYASRTGATAEQAAGWLADLVARGRYVEDVYAAG

Indole is a common intermediate generated by the shikimate-anthranilate pathway to tryptophan and by the metabolism of tryptophan via tryptophanase. Increased production of indole may be achieved by using a cell carrying a 3-deoxy-D-arabino-heptulosonic acid 7-phosphate (DAHP) synthase, encoded by the aroH gene, that is insensitive to tryptophan feedback-inhibition. Suitable tryptophan feedback-insensitive DAHP synthase enzymes are known and described in the art. Suitable tryptophan feedback-insensitive DAHP enzymes are known and described in the art. See, for example, Niu et al. (Niu, H, et al. Metabolic engineering for improving L-tryptophan production in Escherichia coli. J Indust Microbiol Biotechnol 46:55-65 (2019)) and Ger et al. (Ger et al. A single Ser-180 mutation desensitizes feedback inhibition of the phenylalanine-sensitive 3-deoxy-D-arabino-heptulosonae-7-phosphate (DAHP) synthase in Escherichia coli. J Biochem 116:989-990 (1994)).

Construction of a recombinant host cell with the following genetic modifications is expected to produce indol-3-one from glucose and express the PsPCP polypeptide such that the recombinant host cell will produce the pink/red colored PsPCP-indol-3-one complex: (i) introduction of an exogenous polynucleotide encoding a pink chromogenic protein (PsPCP) as described herein; (ii) replacement of the native aroH DAHP synthase gene with a polynucleotide encoding a feedback-insensitive DAHP synthase; (iii) de-repression or overexpression of the genes comprising the tryptophan biosynthetic pathway; (iv) introduction of an exogenous polynucleotide encoding the CYP102A cytochrome P450 monooxygenase from Streptomyces cattleya; and (v) introduction of an exogenous polynucleotide encoding an indoxyl dehydrogenase or reductase.

Construction of a recombinant host cell with the following genetic modifications is expected to produce indol-3-one from indole or tryptophan and express the PsPCP polypeptide such that the recombinant host cell will produce the pink/red PsPCP-indol-3-one complex: (i) introduction of an exogenous polynucleotide encoding a pink chromogenic protein (PsPCP) as described herein; (ii) introduction of an exogenous polynucleotide encoding a tryptophanase; (iii) introduction of an exogenous polynucleotide encoding a CYP102A cytochrome P450 monooxygenase from Streptomyces cattleya, and (iv) introduction of an exogenous polynucleotide encoding an indoxyl dehydrogenase or reductase.

1.-10. (canceled)

11. A meat substitute comprising:
a non-meat protein, and
a pigment composition comprising an aqueous extract of Pleurotus djamor or Pleurotus salmoneostramineus.

12.-14. (canceled)

15. The meat substitute of claim 11, wherein the aqueous extract comprises
a pink chromogenic protein (PCP) in an amount effective for increasing the red color of a meat substitute.

16. The meat substitute of claim 15, wherein the PCP has an absorbance maximum between 450 nm and 600 nm.

17. (canceled)

18. The meat substitute of claim 15, wherein the composition additionally comprises indol-3-one in a molar ration between 0.5:1 to 2:1 with the PCP.

19. The meat substitute of claim 15, wherein the PCP comprises a sequence at least 80% identical to SEQ ID NO:1.

20. The meat substitute of claim 11, wherein the non-meat protein is a plant-based protein selected from the group consisting of pea protein, soy protein, corn protein, and wheat protein.

21. (canceled)

22. The meat substitute of claim 11, wherein the meat substitute comprises 0.01% to 6% by weight of indol-3-one bound PCP.

23. The meat substitute of claim 11, wherein the red color of the meat substitute decreases when heated at 130° C. for 2 minutes.

24. The meat substitute of claim 11, wherein when heated at 130° C. for 2 minutes the a* value of L*a*b* colorimetry of the meat substitute decreases by at least 20%.

25. A method for increasing the red color of a meat substitute, comprising:
adding a pink oyster mushroom extract to a meat substitute comprising a non-meat protein.

26. The method of claim 25, wherein the pink oyster mushroom extract is an aqueous extract of Pleurotus djamor or Pleurotus salmoneostramineus.

27.-29. (canceled)

30. The method of claim 25, wherein the extract comprises a PCP in an amount effective to increase red color of the meat substitute and wherein the PCP has an absorbance maximum between 450 nm and 600 nm and is from a Pleurotus species.

31. (canceled)

32. (canceled)

33. The method of claim 30, wherein the meat substitute comprises indol-3-one and PCP in a molar ratio between 0.5:1 and 2:1.

34. The method of claim 30, wherein the PCP comprises a sequence at least 80% identical to SEQ ID NO:1.

35. A method for decreasing the red color of a cooked meat substitute, comprising:
cooking a meat substitute comprising a pink oyster mushroom extract and a non-meat protein, whereby the red color of the cooked meat substitute is reduced relative to the red color of the meat substitute prior to cooking.

36. The method of claim 35, wherein the pink oyster mushroom extract is an aqueous extract of Pleurotus djamor or Pleurotus salmoneostramineus.

37.-39. (canceled)

40. The method of claim 35, wherein the extract comprises a PCP in an amount effective to increase the red color of the meat substitute prior to cooking and wherein the PCP has an absorbance maximum between 450 nm and 600 nm and is from a Pleurotus species.

41. (canceled)

42. The method of claim 40, wherein the meat substitute comprises indol-3-one and the PCP in a ratio molar ratio between 0.5:1 and 2:1.

43. The method of claim 40, wherein the PCP comprises a sequence at least 80% identical to SEQ ID NO:1.

44. The method of claim 35, wherein when the meat substitute is cooked by heating at 130° C. for 2 minutes the a* value of L*a*b* colorimetry of the meat substitute decreases by at least 20 50%.

45. The method of claim 35, wherein the non-meat protein is or comprises a plant-based protein selected from the group consisting of pea protein, soy protein, corn protein, and wheat protein.

46. (canceled)

47. The method of claim 35, wherein the meat substitute comprises 0.01% to 6% by weight of indol-3-one bound PCP.

48.-59. (canceled)
